(19) United States
(12) Patent Application Publication
(10) Pub. No.: US 2015/0329888 A1
(43) Pub. Date: Nov. 19, 2015

(54) PROCESS FOR THE PRODUCTION OF SOLUTIONS OF SUGARS AND ALCOHOLS FROM LIGNOCELLULOSIC BIOMASS WITH COMPLEMENTARY TREATMENT OF SOLID RESIDUE WITH A HYDRATED INORGANIC SALT

(71) Applicant: IFP ENERGIES NOUVELLES, Rueil-malmaison (FR)

(72) Inventors: Christophe VALLEE, Sassenage (FR); Didier BERNARD, Lyon (FR); Caroline AYMARD, Lyon (FR)

(73) Assignee: IFP Energies nouvelles, Rueil-Malmaison Cedex (FR)

(21) Appl. No.: 14/651,811

(22) PCT Filed: Nov. 19, 2013
(86) PCT No.: PCT/FR2013/052792

§ 371 Date: Jun. 12, 2015

(30) Foreign Application Priority Data
Dec. 14, 2012 (FR) 12/03425

(51) Int. Cl. C12P 19/14; C12P 19/02; C12P 7/10

(52) U.S. Cl. CPC C12P 19/14; C12P 7/10; C12P 19/02; C12P 2201/00; C12P 2203/00

(57) ABSTRACT

The present invention describes a process for the production of solutions of sugars and alcohols from lignocellulosic biomass, comprising a pre-treatment, a step for enzymatic hydrolysis and alcoholic fermentation, a step for extraction of the alcohol produced, a step for separation of the solid residue, an optional step for washing and drying, followed by a step for digestion of the solid residue in a medium comprising at least one hydrated inorganic salt with formula MXₙ.n′H₂O, in which M is a metal selected from groups 1 to 13 of the periodic classification, X is an anion and n is a whole number in the range 1 to 6 and n′ is in the range 0.5 to 12, in order to obtain a solid fraction and a liquid fraction, said solid fraction then being separated and undergoing either an enzymatic hydrolysis step or steps for enzymatic hydrolysis and alcoholic fermentation.

### FIELD OF THE INVENTION

The present invention fits into the context of processes for the production of solutions of sugars and alcohols known as “second generation” processes starting from lignocellulosic biomass. More particularly, it relates to a process for the production of solutions of sugars and ethanol.

### PRIOR ART

Faced with pollution increases and global warming, many studies are currently being carried out with the aim of using and optimizing renewable bioresources such as lignocellulosic biomass.

Lignocellulosic biomass is composed of three principal constituents: cellulose (35% to 50%), hemicellulose (23% to 32%), which is a polysaccharide essentially constituted by pentoses and hexoses, and lignin (15% to 25%), which is a macromolecule with a complex structure and a high molecular weight deriving from the copolymerization of phenylpropenoic alcohols. These various molecules are responsible for the intrinsic properties of the plant wall and are organized into a complex network.

Cellulose, which is in the majority in this biomass, is thus the most abundant polymer on Earth and that which has the greatest potential for forming materials and biofuels. However, the potential of cellulose and its derivatives has not so far been fully exploited, mainly because of the difficulty of extracting the cellulose. In fact, this step is made difficult by the structure of the plants themselves. The main technological stumbling blocks identified for extraction and transformation of cellulose are its accessibility, its crystallinity, its degree of polymerization and the presence of hemicellulose and lignin.

The principle of the process for the conversion of lignocellulosic biomass using biotechnological processes uses a step for enzymatic hydrolysis of cellulose contained in the plant material to produce glucose. The glucose obtained may then be fermented into various products such as alcohols (ethanol, 1,3-propanediol, 1-butanol, 1,4-butanediol etc.) or acids (acetic acid, lactic acid, 3-hydroxypropionic acid, fumaric acid, succinic acid, etc.).

Cellulose and possibly hemicelluloses are the targets for enzymatic hydrolysis, but they are not directly accessible to enzymes. For this reason, these substrates have to undergo a pre-treatment preceding the enzymatic hydrolysis step. The pre-treatment is intended to modify the physical and physico-chemical properties of the lignocellulosic material with a view to improving accessibility to the cellulose trapped in the lignin and hemicellulose matrix.

With a view to converting the substrate into monomeric sugars, an enzymatic hydrolysis is then carried out on the pre-treated substrate. It is carried out with the aid of enzymes produced by a microorganism. The enzymatic solution added to the pre-treated substrate contains enzymes which decompose the cellulose into solutions of sugars containing glucose in particular.

However, the cost of the enzymatic solution penalises processes for the conversion of lignocellulosic biomass using fermentation routes.

One possibility for limiting the overall cost is to reduce the enzymatic load. The hydrolysis yield for the cellulose and hemicelluloses is dependent on the conditions employed, and in particular on the quantity of enzymatic solution added. This dependency is not linear. In fact, a portion of the polymeric sugars is readily hydrolysable following pre-treatment. For his reason, a small dose of enzymes can mean that the expensive enzymatic solution can be used more cost-effectively (kg of sugars hydrolysed per kg of solution used). However, this occurs to the detriment of the hydrolysis yield for the polymeric sugars. It is important to note that the cost of the initial biomass also has a bearing on the cost price of the final product.

In contrast, a maximized hydrolysis of the substrate would necessitate a very high dose of enzymes. It should be noted that certain pre-treatments and/or substrates produce pre-treated solids containing cellulose which is known as recalcitrant, which is difficult to hydrolyse completely.

In order to be economically viable, a process for the production of solutions of sugars and alcohols from lignocellulosic biomass must be a compromise between cost-effective use of the enzymatic solution and upgrading the biomass.

The sugars produced by the enzymatic hydrolysis are then transformed into alcohols by fermentation.

At the end of the steps for hydrolysis and fermentation, an effluent is obtained containing a liquid fraction containing unfermented alcohol(s) and sugars, and a solid residue containing more polymeric sugars in greater or lesser quantities depending on the performances of the enzymatic hydrolysis.

More particularly, the present invention proposes upgrading solid residues obtained from hydrolysis and fermentation steps with a view to improving the overall mass balance and thus the economic viability of the process, in particular on refractory substrates. By recycling solutions containing enzymes and sugars and/or alcohol(s) produced in the various steps of the process, it can also exploit the residual enzymatic activity and obtain a more concentrated solution of sugars and/or alcohol at the end of the process.

A process for transforming lignocellulosic biomass into fermentable sugars with excellent yields has been described in the Applicant's applications FR2963008, FR2963009 and FR11/02730. That process involves digesting biomass in hydrated inorganic salts, which are cheap reagents that are widely available and can be recycled. This technology is simple to apply and can readily be extrapolated to an industrial scale. The cellulose obtained from this treatment is highly reactive in enzymatic hydrolysis.

However, compositional analyses carried out on the solid fraction obtained from this pre-treatment show that the hemicellulose contained in the biomass is partially hydrolysed during digestion. The products resulting from this hydrolysis are thus found in the liquid fraction constituted by anti-solvent and hydrated inorganic salt. Upgrading these hemicellulose hydrolysis products proves to he difficult because of the high concentration of salt in this solution and necessitates a complex and expensive process. Furthermore, recycling of the inorganic salt is made more complex and necessitates a high purge ratio in order to limit the accumulation of hemicellulose hydrolysis products during recycling.

Digestion in hydrated inorganic salts could thus advantageously be carried out on the solid residue from the hydrolysis and fermentation steps, which solid is depleted in hemicelluloses. Using solid residue instead of native biomass in the salt treatment means that recycling of the salt is facilitated.

The aim of the present invention is to propose a process for the complementary treatment of solid residue obtained after the hydrolysis and fermentation steps in order to allow optimized upgrading of the initial resource. The complementary treatment process of the invention is a treatment with hydrated inorganic salts.

### DETAILED DESCRIPTION OF THE INVENTION

The present invention describes a process for the production of solutions of sugars and alcohols from lignocellulosic biomass, comprising the following steps:

a) carrying out a pre-treatment of a feed comprising lignocellulosic biomass,

b) carrying out an enzymatic hydrolysis of the pre-treated and optionally washed feed obtained from step a) using cellulolytic and/or hemicellulolytic enzymes, producing an effluent comprising a hydrolysate containing sugars and a solid residue,

c) carrying out an alcoholic fermentation of the hydrolysate contained in the effluent obtained from step b) into alcohol with an alcoholigenic microorganism in order to produce a fermented effluent containing alcohol,

d) carrying out a step for extracting alcohol from the fermented effluent obtained from step c),

e) carrying out a step for separating the effluent obtained either at the end of step c) or at the end of step d) in order to produce a solid residue and a liquid fraction,

f) carrying out an optional step for washing and an optional step for drying the solid residue obtained in step e),

g) carrying out a step for digesting the solid residue obtained in step e) and/or f) in a medium comprising at least one hydrated inorganic salt with formula MXₙ.n′H₂O, in which M is a metal selected from groups 1 to 13 of the periodic classification, X is an anion and n is a whole number in the range 1 to 6 and n′ is in the range 0.5 to 12, in order to obtain a solid fraction and a liquid fraction,

h) carrying out a step for separating the solid fraction and the liquid fraction obtained in step g),

i) carrying out an optional step for treatment of said solid fraction obtained in step h),

j) carrying out either a step for enzymatic hydrolysis of said solid fraction obtained in step h) and/or i), or steps for enzymatic hydrolysis and alcoholic fermentation of said solid fraction obtained in step h) and/or i).

The process of the present invention can be used to efficiently transform various types of native lignocellulosic biomass into alcohol(s) and into one or more solutions of sugars, with optimized extraction of the cellulose fraction. It also has the advantage of using cheaper reagents which are widely available and capable of being recycled, meaning that the supplemental processing cost is low. This technology is also simple to carry out and means that scaling up to industrial scale can readily be envisaged.

In this manner, the process of the invention produces alcohols and/or sugars by means of steps d), e), j) and optionally f). In addition, a solution of sugars obtained from the hemicellulose obtained after step a) is obtained if an acid pre-treatment or treatment without adding chemical reagents is carried out, supplemented by a solid/liquid separation and optionally by washing before step b).

The lignocellulosic biomass or lignocellulosic materials employed in the process of the invention is obtained from wood (hardwood or softwood), which may be raw or treated, from agricultural by-products such as straw, plant fibres, forest cultures, alcoholigenic, sugar-producing and cereal-producing plant residues, paper industry residues, marine biomass (for example cellulosic macroalgae) or transformation products of cellulosic or lignocellulosic materials. The lignocellulosic materials may also be biopolymers and are preferably rich in cellulose.

Preferably, the lignocellulosic biomass used is wood, wheat straw, wood pulp, miscanthus, rice straw or corn stalks.

In accordance with the process of the present invention, the various types of lignocellulosic biomass may be used alone or as a mixture.

The various steps of the process will now be described in detail.

### Pre-Treatment (Step a)

The step for pre-treatment of the feed comprising lignocellulosic biomass can be used to improve the susceptibility of the cellulose to enzymatic hydrolysis.

The pre-treatment step in accordance with step a) of the process of the invention may be carried out using any of the lignocellulosic biomass pre-treatment types known to the skilled person. A prior conditioning step including, for example, grinding or de-stoning, may also be carried out. The pre-treatment step may be a heat, chemical, mechanical and/or enzymatic treatment or a combination of these treatments.

In accordance with a preferred variation, the pre-treatment step is selected from a pre-treatment under acid conditions such as an acid digestion or steam explosion under acid conditions, a pre-treatment in alkali media such as a sodium sulphide pre-treatment (Kraft process), an ARP (Ammonia Recycle Percolation) process or an AFEX (Ammonia Fibre Explosion) process, an oxidizing pre-treatment such as a pre-treatment using ozone, hydrogen peroxide, oxygen or peracetic acid, a pre-treatment without the addition of chemical reagents, such as steam explosion without adding acid or a very hot water wash pre-treatment, or indeed an organosolv process.

In accordance with a particularly preferred variation, the pre-treatment step is carried out by steam explosion, with or without the addition of acid.

The pre-treatment efficiency is measured both by the material balance at the end of pre-treatment (degree of recovery of sugars in the form of soluble monomers or oligomers or solid polymers) and also by the susceptibility of the cellulosic and hemicellulosic residues to hydrolysis.

For the acid pre-treatments, the pre-treated solid is mainly constituted by cellulose and lignin, the vast majority of the hemicelluloses having been hydrolysed under acid conditions.

For the delignifying pre-treatments, for example under alkaline conditions, the oxidizing pre-treatments or the organosolv type treatments, the solid is mainly constituted by cellulose and hemicelluloses.

For the AFEX pre-treatment, the pre-treated solid has a composition which is very similar to the feed.

For the pre-treatments without the addition of chemical products, for example steam explosion without acid or hot water washing, the solid is depleted in hemicellulose compared with the native biomass, but nevertheless retains a non-negligible portion.

The pre-treatments under acid conditions, for example acid digestion or steam explosion with acid impregnation, tend to form degradation products of the pentose and hexose sugars, for example furfural, or 5-HMF. The formation of these degradation products increases with the severity of the pre-treatment (heat, dwell time, acidity). The hemicellulose of the lignocellulosic substrate is hydrolysed very easily under acid conditions and at high temperature. However, too severe a pre-treatment risks not acting sufficiently on the substrate; this would reduce its susceptibility to enzymatic hydrolysis.

Dilute acid pre-treatment is generally carried out in the presence of dilute sulphuric acid or hydrochloric acid in a proportion of 0.5% to 10% with respect to the substrate dry matter. Two methods are used: a first at a temperature of >60° C., which is continuous, which is suitable for low dry matter feeds (5% to 10%), and a second, batch method which is carried out at a temperature which is frequently <150° C., which can be used for dry matter concentrations in the range 10% to 40%. The higher the temperature, the greater is the loss of dry matter. The dwell times are dependent on the temperature used.

Pre-treatment by explosion with steam is also known as steam explosion, steam gunning, explosive decompression and steam pre-treatment. The plant matter is heated rapidly to a high temperature (150° C.-250° C.) by injecting steam under pressure. The treatment is generally stopped by a sudden decompression, known as decompression or explosion, which breaks down the lignocellulosic matrix. The dwell times vary from 10 seconds to several minutes for pressures of 10 to 50 bar. This technique is carried out either in batches or continuously. Certain technologies propose injecting water in order to cool the medium before decompression. Steam explosion may be preceded by acid impregnation in order to increase hydrolysis of the hemicelluloses during digestion. When steam explosion is applied to a substrate which has already been acidified, for example with H₂SO₄, it results in dissolution and almost complete hydrolysis of the hemicelluloses into their monomers, thereby limiting degradation into furfural. Furthermore, the susceptibility of the cellulose to enzymatic hydrolysis is improved. The use of an acid catalyst means that the temperature of the process can be reduced (150° C. to 200° C. as opposed to 250° C. for steam explosion with no catalyst), and thus the formation of degradation compounds can be minimized.

The pre-treatments in alkaline media have the advantage of generating far fewer polysaccharide degradation products. They represent a viable alternative to acid pre-treatments, although their cost is currently higher, in particular for the chemical products employed. Examples of alkaline medium pre-treatments are a pre-treatment with a mixture of sodium sulphide and sodium hydroxide, also known as the Kraft process, which is conventionally used in processes for the production of paper products known as Kraft or “sulphate pulping” processes, at the end of which paper pulp is obtained, Ammonia Fibre Explosion pre-treatment, also known as AFEX, or a pre-treatment by percolation using ammonia with a recycle, also known as ARP pre-treatment (Ammonia Recycle Percolation).

Certain physico-chemical pre-treatments do not involve the addition of chemical reagents. In fact, at high temperature and in the presence of water, certain ester functions present in the hemicelluloses will hydrolyse, slightly acidifying the medium, which catalyses the partial hydrolysis of the sugars present in the hemicellulose. An example which may be cited is steam explosion pre-treatment without the addition of acid described above, and very hot water pre-treatment. The absence of chemical reagents renders these pre-treatments less expensive, but the reactivity of such pre-treated cellulose in enzymatic hydrolysis is reduced.

Originating in the paper industry, the Organosolv process consists of dissolving and extracting lignin and hemicelluloses in an organic solvent (in general methanol or ethanol). An acid catalyst (HCl or H₂SO₄) is often added when the temperature used is less than 185° C. The organic solvent is then extracted by evaporation and recycled.

Other pre-treatments are also being studied; a non-exhaustive review has been carried out by Ogier et al., 1999, Oil & Gas Science and Technology—IFP review, Vol. 54 (1999), No. 1, pp. 67-94 or more recently in Biotechnology Advances, Volume 29, Issue 6, November-December 2011, Pages 675-685 and Biofuels, Bioproducts and Biorefining Volume 6, Issue 5, pages 561-579, September/October 2012.

In a variation of the process, the pre-treated feed may undergo a step for solid/liquid separation then may optionally be washed, preferably with water, before the enzymatic hydrolysis step.

A pre-treatment under acid conditions or without chemical reagents results in partial or almost complete hydrolysis of the hemicelluloses, principally into monomeric sugars (pentoses and hexoses) and soluble oligomers, depending on the type of biomass.

When the pre-treatment of step a) is carried out under acid conditions or without chemical reagents, before the enzymatic hydrolysis step b), the pre-treated feed may undergo a separation step in order to recover a solid fraction and a liquid fraction. At least a portion of the solid fraction is then sent to the enzymatic hydrolysis step b) and at least a portion of the liquid fraction may be recycled to the enzymatic hydrolysis step j). In fact, recycling at least a portion of the liquid fraction containing the sugars obtained from the hemicellulose means that a more concentrated solution of sugars can be obtained at the end of the process.

### Enzymatic Hydrolysis and Alcoholic Fermentation (Steps b) and c)

The conversion of cellulose into alcohol comprises at least one step for enzymatic hydrolysis of the cellulose into glucose and a step for fermentation of glucose into alcohol, these two steps possibly being carried out separately or simultaneously. When the two steps are operated simultaneously, the process is known as “SSF” (Simultaneous Saccharification and Fermentation). The hydrolysis and fermentation steps may also be carried out using other arrangements which are known to the skilled person, such as the “PSSF” (Presacchararification followed by Simultaneous Saccharification and Fermentation) process, or indeed the “HHF” (Hybrid Hydrolysis and Fermentation) process.

Step b) for enzymatic hydrolysis and step c) for alcoholic fermentation of the process of the invention are carried out separately or simultaneously. In accordance with a variation of the process of the invention, steps b) and c) are carried out in two separate reactors. In accordance with another variation of the process of the invention, steps b) and c) are carried out in the same reactor.

In accordance with step b) of the process of the invention, the pre-treated and optionally washed feed obtained from step a) is sent to an enzymatic hydrolysis step in order to convert the pre-treated feed into monomeric sugars. The enzymatic hydrolysis step uses cellulolytic and/or hemicellulolytic enzymes and produces an effluent comprising a hydrolysate containing sugars and a solid residue which is insoluble in water.

The enzymatic hydrolysis is carried out at a pH in the range 4.5 to 5.5, preferably in the range 4.8 to 5.2. It is generally carried out at a temperature in the range 40° C. to 60° C. The enzymatic hydrolysis is carried out using enzymes produced by a microorganism. The enzymatic solution added to the pre-treated substrate contains enzymes which decompose the cellulose into sugars.

Microorganisms such as fungi belonging to the genera Trichoderma, Aspergillus, Penicillium or Schizophyllum, or anaerobic bacteria belonging to the genus Clostridium, for example, produce these enzymes, in particular containing cellulases and hemicellulases, adapted to the intense hydrolysis of cellulose and hemicelluloses.

Highly preferably, the cellulolytic and/or hemicellulolytic enzymes of step b) are produced by the microorganism Trichoderma reesei.

The enzymatic hydrolysis is carried out with an enzymatic solution often produced from a filamentous fungus such as Trichoderma reesei, for example, or occasionally by Aspergillus niger. These fungi secrete an “enzymatic cocktail” composed of several different enzymes, up to 50, such as CBHI or CBHII, for example, which are involved in the hydrolysis of cellulose, and xylanases which are involved in the hydrolysis of hemicelluloses. The exact composition of the cocktail depends on the strain of fungus used and the culture conditions.

The sugars obtained by enzymatic hydrolysis are then fermented to form alcohols such as ethanol, 1,3-propanediol, isopropanol, 1-butanol, isobutanol or 1,4-butanediol, alone or as a mixture. Preferably, the alcoholic fermentation carried out in step c) produces ethanol.

The alcoholic fermentation of step c) is carried out using yeasts or other alcoholigenic microorganisms. In the context of the present invention, the term “alcoholic fermentation” designates a process for the fermentation of sugars into alcohol(s) using microorganisms alone.

The alcoholigenic microorganisms used during the step for alcoholic fermentation of hexoses are preferably selected from yeasts and bacteria, optionally genetically modified.

When the alcoholigenic microorganism is a yeast, Saccharomyces cerevisiae is that which performs the best. It is also possible to select yeasts such as Schizosaccharomyces pombe or Saccharomyces uvarum or diastaticus. More thermophilic yeasts such as Kluyveromyces fragilis (now often given the designation K. marxianus) are also of interest, in particular when the enzymatic hydrolysis and alcoholic fermentation are carried out simultaneously (SSF process).

A genetically modified organism such as, for example, a yeast of the Saccharomyces cerevisiae type such as TMB 3400 (Ohgren et al, J. of Biotech 126, 488-498, 2006) may also be used. This yeast can be used to ferment a portion of the pentoses into ethanol during the step for ethyl fermentation of hexoses when glucose is in a limiting concentration.

When the alcoholigenic microorganism is a bacterium, Zymomonas mobilis is preferred; it has an effective assimilation pathway for the production of ethanol, or anaerobic bacteria of the genus Clostridium such as, for example, Clostridium acetobutylicum for the production of mixtures of alcohols and solvents such as acetone-butanol-ethanol (ABE) or isopropanol-butanol-ethanol (IBE), or indeed Escherichia coli for the production of isobutanol, for example.

The alcoholic fermentation is preferably carried out at a temperature in the range 30° C. to 40° C., and at a pH in the range 3 to 6.5.

Yeasts, and preferably Saccharomyces cerevisiae, are the microorganisms which are most preferably used. They are more robust, safe and do not require sterility of the facilities to carry out the process.

Yeasts of the genus Saccharomyces are capable of fermenting hexoses alone and exclusively (essentially glucose and mannose). These yeasts upgrade the hexoses in an optimized manner into ethanol and can be used to obtain conversion yields of the order of 0.46 (w/w) to 0.48 (w/w), which is close to the maximum theoretical yield which is 0.51 (w/w). Only the pentoses and a few marginal carbonaceous sources are not used by these yeasts.

When enzymatic hydrolysis and alcoholic fermentation are carried out in one and the same operation (SSF process), the temperature is in the range 30° C. to 45° C. and the pH is in the range 4 to 6.

### Extraction of Alcohol (Step d)

In accordance with step d) of the process of the invention, a step for extracting alcohol from the fermented effluent obtained from step c) is carried out. This extraction step generally comprises at least one distillation step.

### Solid/Liquid Separation (Step e)

In accordance with step e) of the process of the invention, a step for separation of the effluent obtained is carried out either at the end of the fermentation step c) or at the end of the alcohol extraction step d) in order to produce a solid residue and a liquid fraction.

This separation step may be carried out using the usual solid-liquid separation techniques, for example by settling, by filtration or by centrifuging.

This separation can be used to obtain a solid residue containing upgradable polymeric sugars and a liquid fraction (also termed stillage) containing unfermented sugars.

In a variation, at least a portion of the liquid fraction obtained in step e) is recycled to the enzymatic hydrolysis step j). As an example, when step e) is carried out before the extraction step d), the liquid fraction obtained from step e) contains one or more alcohol(s). Recycling at least a portion of this liquid fraction to step j) means that the final titre of alcohol(s) can be increased when an enzymatic hydrolysis and a fermentation are carried out in step j).

The proportion of each component in the extracted solid residue is a function of the initial substrate, the type of pre-treatment carried out and the operating conditions for the enzymatic hydrolysis and the fermentation.

In the case of a steam explosion type pre-treatment under acid conditions, the quantity of lignin is in the range 20% to 90% by weight, and more preferably in the range 30% to 85% by weight, the quantity of cellulose is in the range 10% to 70% by weight, preferably in the range 20% to 60% by weight, and the quantity of hemicelluloses is in the range 0 to 30% by weight, preferably in the range 1% to 10% by weight.

For the alkaline type pre-treatments, as an example, the composition of the solid residue will be:
in the case of a Kraft type de-lignifying pre-treatment, the quantity of lignin is in the range 2% to 60% by weight, and more preferably in the range 3% to 50% by weight, the quantity of cellulose is in the range 10% to 70% by weight, preferably in the range 20% to 60% by weight, and the quantity of hemicelluloses is in the range 1% to 50% by weight, preferably in the range 2% to 40% by weight,
in the case of a partially delignifying pre-treatment, of the ARP type, the quantity of lignin is in the range 5% to 75% by weight, and more preferably in the range 5% to 60% by weight, the quantity of cellulose is in the range 10% to 75% by weight, preferably in the range 20% to 65% by weight and the quantity of hemicellulose is in the range 1% to 60% by weight, preferably in the range 2% to 45% by weight,
in the case of a non-delignifying alkaline pre-treatment such as AFEX, for example, the quantity of lignin is in the range 10% to 80%, and more preferably in the range 10% to 65%, the quantity of cellulose is in the range 4% to 65%, preferably in the range 10% to 60%, and the quantity of hemicelluloses is in the range 1% to 60%, preferably in the range 2% to 50%.

In the case of a steam explosion type pre-treatment without the addition of acid (pre-treatment with no chemical reagents), the quantity of lignin is in the range 20% to 90% by weight, and more preferably in the range 25% to 85% by weight, the quantity of cellulose is in the range 10% to 70% by weight, preferably in the range 20% to 60% by weight, and the quantity of hemicelluloses is in the range 1% to 50% by weight, preferably in the range 2% to 40% by weight.

Preferably, in order to carry out the invention, the pre-treatments and operating conditions for the enzymatic hydrolysis will be selected so as to obtain a solid residue in step e) containing less than 30% by weight of the hemicelluloses contained in the biomass introduced into step a), preferably less than 20% by weight, and particularly preferably less than 10% by weight.

### Washing and Drying (Optional Step f)

In accordance with step f) of the process of the invention, an optional wash and optional drying of the solid residue obtained in step e) is carried out.

Washing is carried out in co-current or counter-current mode, optionally in several stages. Washing is carried out with water or a stream containing mainly water. Preferably, it is carried out with water.

In accordance with a preferred variation, at least a portion of the washing solution obtained in the step for washing the solid residue f) is recycled to the enzymatic hydrolysis step j). As an example, when step e) is carried out before the extraction step d) and a washing step f) is carried out on the solid residue obtained in step e), the washing solution contains one or more alcohol(s). Recycling at least a portion of this washing solution to step j) means that the final titre of alcohol(s) can be increased when an enzymatic hydrolysis and a fermentation are carried out in step j).

The washed solid residue may optionally be compressed in order to increase the percentage of dry matter contained in the solid.

In a variation, the optionally washed solid residue is then dried. The drying step may be carried out using any of the processes known to the person skilled in the art, such as evaporation, for example. Examples of known technologies for drying by evaporation are the rotary furnace, moving bed, fluidized bed, heated auger, and contacting with metal beads providing heat. These technologies may optionally use a co- or counter-current of moving gas such as nitrogen, or any other gas which is inert under the reaction conditions.

The drying step is carried out at a temperature of 20° C. or more.

At the end of the drying step, the residual water content is less than 30% by weight, preferably less than 20% by weight and still more preferably less than 10% by weight.

### Digestion of Solid Residue in a Medium Comprising at Least One Hydrated Inorganic Salt (Step g)

The step for digestion with hydrated inorganic salts can be used to obtain a solid fraction which contains the major portion of the cellulose present in the solid residue obtained from step f). This cellulose has the property of being particularly reactive in enzymatic hydrolysis. A liquid fraction containing the hydrated inorganic salt or salts is also obtained.

Digestion of the solid residue obtained from step e) and/or f) is carried out in the presence of a hydrated inorganic salt with formula (I): MXₙ.n′H₂O in which M is a metal selected from groups 1 to 13 of the periodic classification, X is an anion, and n is a whole number in the range 1 to 6, and n′ is in the range 0.5 to 12.

A mixture of hydrated inorganic salts may be used to digest the solid residue obtained from step e) and/or f).

The anion X may be a monovalent, divalent or trivalent anion. Preferably, the anion X is a halide anion selected from Cl⁻, F⁻, Br⁻ and I⁻, a perchlorate anion (ClO₄⁻), a thiocyanate anion (SCN⁻), a nitrate anion (NO₃⁻), a para-methylbenzene sulphonate anion (CH₃—C₆H₄—SO₃⁻), an acetate anion (CH₃COO⁻), a sulphate anion (SO₄²⁻), an oxalate anion (C₂O₄²⁻) or a phosphate anion (PO₄³⁻). More preferably, the anion X is a chloride.

Preferably, the metal M in formula (I) is selected from lithium, iron, zinc or aluminium.

More particularly, the hydrated inorganic salt is selected from: LiCl.H₂O, LiCl.2H₂O, ZnCl₂.1.5H₂O, ZnCl₂.2.5H₂O, ZnCl₂.4H₂O and FeCl₃.6H₂O. Particularly preferably, the salt is selected from ZnCl₂.1.5H₂O, ZnCl₂.2.5H₂O and ZnCl₂.4H₂O.

Preferably, the digestion temperature is in the range −20° C. to 250° C., preferably in the range 20° C. to 160° C.

When the metal M of the hydrated inorganic salt is selected from groups 1 and 2 of the periodic table, the digestion temperature is preferably in the range 100° C. to 160° C.

When the metal M of the hydrated inorganic salt is selected from groups 3 to 13 of the periodic table, the digestion temperature is preferably in the range 20° C. to 120° C.

The digestion period is in the range 0.5 minute to 168 h, preferably in the range 5 minutes to 24 h, and still more preferably in the range 20 minutes to 12 h.

In accordance with the process of the present invention, several successive digestion steps may be carried out.

The digestion step may be carried out in the presence of one or more organic solvents selected from alcohols such as methanol, ethanol, n-propanol, isopropanol, n-butanol, isobutanol or tert-butanol, diols and polyols such as ethanediol, propanediol or glycerol, aminoalcohols such as ethanolamine, diethanolamine or triethanolamine, ketones such as acetone or methylethylketone, carboxylic acids such as formic acid or acetic acid, dimethylformamide, dimethylacetamide, dimethylsulphoxide, acetonitrile, aromatic solvents such as benzene, toluene, xylenes and alkanes.

In accordance with another embodiment, the digestion step may be carried out in the absence of organic solvent.

Preferably, the digestion medium of step e) is constituted by one or more hydrated inorganic salts.

In the digestion step, the dried solid fraction is present in a quantity in the range 4% to 40% by weight on a dry matter basis of the total mass of the solid/hydrated inorganic salt fraction, preferably in a quantity in the range 5% to 30% by weight.

### Solid/Liquid Separation (Step h)

At the end of the digestion step, a mixture of a solid fraction containing the pre-treated cellulosic substrate and a liquid fraction containing the hydrated inorganic salt or salts and optionally an organic solvent is obtained. This mixture is sent to a solid/liquid separation step h).

The step h) for separation of the solid fraction is preferably carried out by precipitation, by addition of at least one anti-solvent. Adding anti-solvent promotes precipitation of the solid fraction.

The separation step h) may be carried out using the usual solid-liquid separation techniques, for example by settling, filtration or centrifuging.

The anti-solvent used is a solvent or a solvent mixture selected from water, alcohols such as methanol, ethanol, n-propanol, isopropanol, n-butanol, isobutanol or tert-butanol, diols and polyols such as ethanediol, propanediol or glycerol, aminoalcohols such as ethanolamine, diethanolamine or triethanolamine, ketones such as acetone or methylethylketone, carboxylic acids such as formic acid or acetic acid, esters such as ethyl acetate or isopropyl acetate, dimethylformamide, dimethylacetamide, dimethylsulphoxide or acetonitrile.

Preferably, the anti-solvent is selected from water, methanol or ethanol.

Highly preferably, the anti-solvent is water, alone or as a mixture, preferably alone.

At the end of the separation step h), a fraction termed a solid fraction and a liquid fraction are obtained.

The solid fraction is composed of solid matter, between 5% and 60%, and preferably between 15% and 45%, and a liquid phase. The presence of liquid in this fraction is linked to limitations in the liquid/solid separation equipment. The solid matter contains the major portion of the cellulose of the solid residue obtained from step e), between 60% and 100%, and preferably between 75% and 99% of the cellulose introduced into the digestion step g).

The liquid fraction contains the hydrated inorganic salt or salts used during the digestion step, and the optional anti-solvent. Because the hemicellulose is eliminated by the steps for pre-treatment a) and enzymatic hydrolysis b), this fraction contains only very little hemicellulose (or products derived from hemicellulose). It may contain lignin.

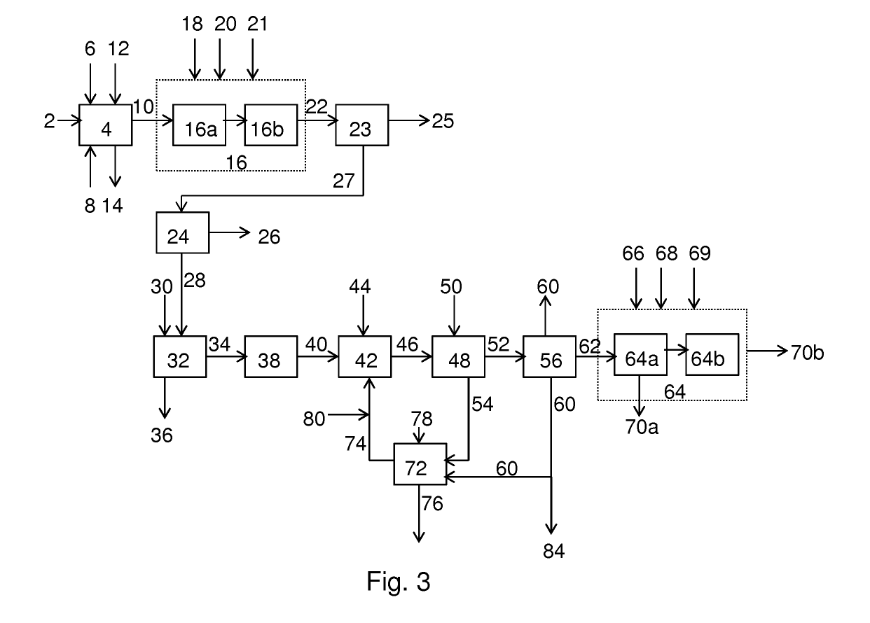

This low organic matter content in the liquid fraction means that recycling of the salts to the digestion step is facilitated and the amount of purge for this recycle is reduced. Variations to the recycle will be described in FIG. 3.

### Supplemental Treatments (Optional Step i)

The solid fraction obtained at the end of the separation step h) may optionally undergo supplemental treatments (step i). These supplemental treatments may in particular be aimed at eliminating traces of hydrated inorganic salts in this solid fraction.

Step i) for the treatment of the solid fraction obtained in step h) may be carried out by one or more washes, by neutralisation, by compression and/or by drying.

The washes may be carried out with anti-solvent or with water. The washes may also be carried out with a stream originating from a unit for the transformation of products obtained from the pre-treatment process of the present invention.

By way of example, when the process of the present invention is used as a pre-treatment upstream of a unit for the production of cellulosic ethanol, the washes may be carried out with a stream originating from this unit for the production of cellulosic ethanol.

Neutralisation may be carried out by suspending the solid fraction obtained in step h) in water and adding a base. The term “base” as used by us here means any chemical species which, when added to water, provides an aqueous solution with a pH of more than 7. Neutralisation may be carried out with an organic or inorganic base. Bases which may be used for the neutralisation which may be cited include sodium hydroxide, potassium hydroxide, sodium carbonate and potassium carbonate, sodium bicarbonate and potassium bicarbonate and ammonia.

The solid fraction obtained at the end of the separation step h) may optionally be dried or compressed in order to increase the percentage of dry matter contained in the solid.

### Enzymatic Hydrolysis Optionally Followed by an Alcoholic Fermentation of the Solid Fraction Obtained After Digestion Using a Hydrated Inorganic Salt (Step j)

In accordance with a first variation of step j) of the process of the invention, a step for enzymatic hydrolysis of the solid fraction obtained in step h) and/or i) is carried out to convert the cellulose contained in the solid fraction into a solution of sugars primarily containing monosaccharides such as glucose.

In accordance with a second variation of step j) of the process of the invention, the steps for enzymatic hydrolysis and alcoholic fermentation of the solid fraction obtained in step h) and/or i) are carried out in order to convert the cellulose contained in the solid fraction into a solution of sugars and to transform the solution of sugars into alcohols.

The enzymatic hydrolysis in this step is carried out in the same manner and in the same ranges as those described for step b). The operating conditions for the enzymatic hydrolysis may be identical or different from those of step b). The enzymatic cocktail introduced in this second enzymatic hydrolysis has a composition which is identical to or different from that of the enzymatic cocktail introduced in the enzymatic hydrolysis of step b).

The alcoholic fermentation in this step is carried out in the same manner and using the same ranges as those described for step c). The operating conditions for the fermentation may be identical to or different from those of step c). The alcoholigenic microorganism introduced in this second alcoholic fermentation may be identical to or different from the alcoholigenic microorganism introduced in the alcoholic fermentation of step c). The fermentation must obtained is then distilled to separate the stillage and the alcohol produced. Preferably, the alcoholic fermentation of step j) produces ethanol.

Step j) for enzymatic hydrolysis and alcoholic fermentation of the process in accordance with the invention are carried out separately or simultaneously (SSF process).

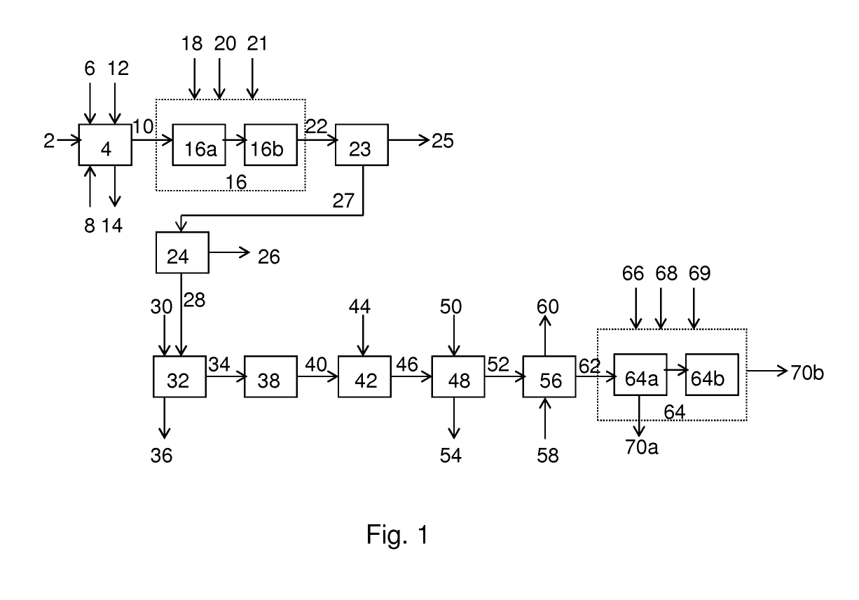

A preferred variation of the process will now be described with reference to FIG. 1.

The lignocellulosic biomass is introduced into the reactor 4 via the line 2 in which the pre-treatment step a) takes place. The pre-treatment step a) may be carried out using any of the techniques known to the skilled person; preferably, steam explosion pre-treatment is carried out, with or without adding acid. The reagent or reagents which may be required are introduced via the line 6. The necessary water and/or steam are optionally introduced via the line 8. At the end of the pre-treatment step, a pre-treated feed is withdrawn via the line 10.

In the particular case in which the pre-treatment is carried out under acidic or slightly acidic conditions (i.e. without the addition of chemical reagents), either by adding an acid or by liberating acids contained in the hemicelluloses (auto-hydrolysis), such as steam explosion with and without acid, for example, a solid/liquid separation is advantageously carried out following the pre-treatment in order to remove all or a portion of the liquid stream which contains the hemicelluloses dissolved during the pre-treatment using the line 14. Optionally, the line 12 may be used to supply a washing liquid, highly preferably water, in order to improve recovery of the sugars obtained from the hemicelluloses. The stream extracted via the line 14 is a sugar-containing solution. At least a portion of this stream 14 may be recycled to the vessel 64 of the enzymatic hydrolysis step j) (not shown).

The pre-treated feed 10 is then sent to the reactor 16 in which the step b) for enzymatic hydrolysis 16a and the step c) for alcoholic fermentation 16b take place. One or more reactors may be employed, these reactors possibly being similar or having different geometries, with similar or different stirring systems. This step may be carried out in a batch, semi-continuous or continuous mode, or a combination, depending on the reactors. In FIG. 1, the enzymatic hydrolysis and alcoholic fermentation units are represented separately from each other; however, it is possible to have a single reactor in which the hydrolysis and the fermentation are carried out simultaneously (SSF process).

Optionally, water may be added via the line 18 in order to adjust the percentage of dry matter employed during this step. The enzymatic cocktail, in particular containing cellulases and/or hemicellulases, is introduced via the line 20. The alcoholigenic microorganisms used for the alcoholic fermentation are introduced via the line 21. The stream 22 leaving the reactor 16 is a mixture of alcohols, a liquid fraction containing unfermented sugars (known as stillage) and a solid residue which is insoluble in water. The solid residue is in part composed of cellulose and hemicellulose which has not been hydrolysed, and lignin.

Preferably, by the combined action of pre-treatment step a) and enzymatic hydrolysis step b), the solid fraction of the stream 22 contains less than 30% by weight, preferably less than 20% by weight and highly preferably less than 10% by weight of the hemicelluloses initially contained in the biomass introduced into the line 2.

The stream 22 is sent to the alcohol extraction device 23 (step d). The extraction is preferably carried out by distillation and can be used to recover the alcohol via the line 25.

The remaining stream 27 containing the solid residue and the liquid fraction containing the unfermented sugars is sent to the separation device 24. At the end of the separation step e), a liquid fraction is obtained containing unfermented sugars at 26 and the solid residue at 28. At least a portion of the liquid fraction may be recycled to the vessel 64 of the enzymatic hydrolysis step j) (not shown).

The solid residue 28 contains a solid phase and a liquid phase the composition of which is similar to that of the stream 26, due to current limitations in the solid/liquid separation equipment. The solid residue 28 may be sent to a washing step 32 (step f) in which a washing liquid, preferably water, is introduced into the line 30. The washing solution is extracted via the line 36; it contains more than 70%, preferably more than 85% and still more preferably more than 90% of the monomeric sugars (soluble) contained in the stream 28, At least a portion of this washing solution may be recycled to the vessel 64 of the enzymatic hydrolysis step j) (not shown). Optionally, the washed solid residue 34 is sent to a drying device 38 (step f).

The optionally washed and optionally dried solid residue is then introduced into the digestion reactor 42 via the line 40 in which the digestion step g) takes place. The digestion medium comprising one or more hydrated inorganic salts and optionally an organic solvent is introduced via the line 44.

At the end of the digestion step, a mixture containing the treated solid residue, the hydrated inorganic salt or salts and an optional organic solvent is withdrawn via the line 46. This mixture is sent to the liquid/solid separation device 48 in which the separation step h) is carried out. The optional anti-solvent is added via the line 50.

At the end of the separation step h), a solid fraction 52 and a liquid fraction 54 containing the hydrated inorganic salt or salts are obtained.

The solid fraction 52 may optionally undergo supplemental treatments (step i) carried out in the vessel 56.

The agents which may be necessary for the treatment(s) carried out in the vessel 56 are introduced via the line 58. Any residues from this (these) treatment(s) are withdrawn via the line 60.

In accordance with a first variation, the treated solid fraction is withdrawn via the line 62 and is sent to the reactor 64 (step j) in which an enzymatic hydrolysis step 64a is carried out. The enzymatic cocktail is introduced via the line 66; this cocktail may be identical to or different from the enzymatic cocktail 20 employed in step b). Optionally, water may be added via the line 68 to adjust the percentage of dry matter employed during this step. A sugar solution is withdrawn via the line 70a.

In accordance with a second variation, the treated solid fraction is withdrawn via the line 62 and is sent to the reactor 64 (step j) in which an enzymatic hydrolysis step 64a and an alcoholic fermentation step 64b are carried out. The enzymatic cocktail is introduced via the line 66; this cocktail may be identical to or different from the enzymatic cocktail 20 employed in step b). Optionally, water may be added via the line 68 to adjust the dry matter percentage employed during this step. The alcoholigenic microorganisms used for the alcoholic fermentation are introduced via the line 69; these microorganisms have a composition which is identical to or different from the microorganisms 21. The fermentation must obtained is then distilled (not shown) in order to separate the stillage and the alcohol produced (70b).

In this manner, the process of the invention produces alcohols and/or sugars via the steps d) (stream 25), e) (stream 26), j) (stream 70a and b) and optionally f) (stream 36). In addition, a solution of sugars obtained from hemicellulose obtained after step a) (stream 14) is obtained if an acid pre-treatment is carried out or no chemical reagents are added, supplemented by a solid/liquid separation and optionally by washing before step b).

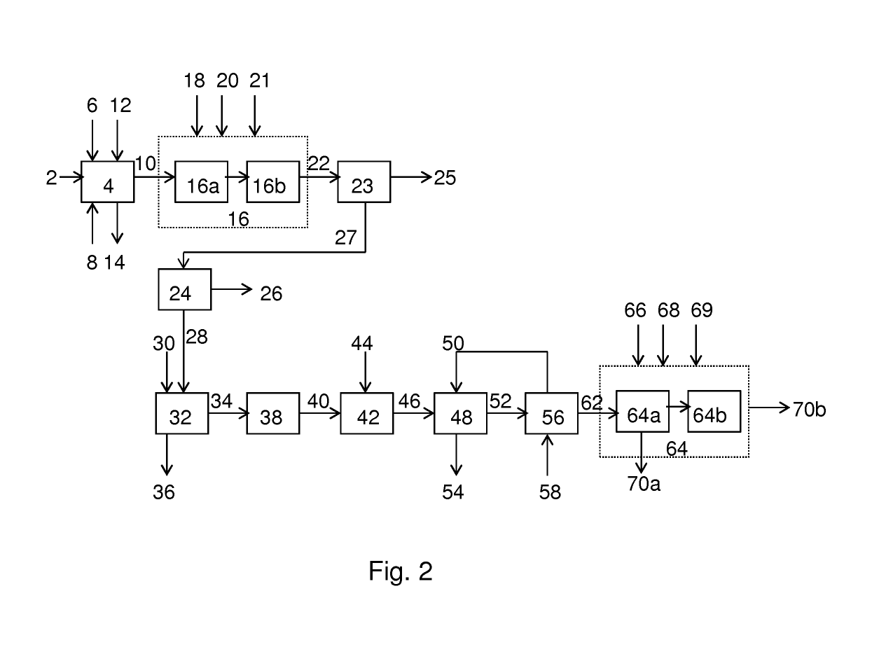

In accordance with the embodiment of FIG. 2, the separation step h) is carried out with the addition of an anti-solvent, and the supplemental treatment carried out in the vessel 56 (step i) is constituted by one or more washes carried out with the anti-solvent introduced via the line 58. After washing, the liquid 50 principally contains the anti-solvent and hydrated inorganic salt. This liquid 50 is used as an anti-solvent in the separation step h). This embodiment can be used to improve the degree of recovery of the hydrated inorganic salt and improve the purity of the solid fraction 62 while limiting the consumption of anti-solvent. Preferably, the anti-solvent is water in this embodiment.

The embodiment of FIG. 3 pertains to recycling the inorganic salt contained in the various liquid fractions obtained during the process.

In a first variation, at least a portion of the liquid fraction obtained in the separation step h) at 54 is sent to a purification step 72, denoted step k), in order to concentrate the inorganic salt contained in the liquid fraction and to obtain a liquid fraction 74 containing the concentrated inorganic salt and another liquid fraction 76 which is depleted in inorganic salt, at least a portion of said liquid fraction 74 containing the concentrated inorganic salt then being recycled to the digestion step g).

The purification step k) may in particular be a step for separation of hydrated inorganic salt and anti-solvent. This separation may be carried out using any of the processes known to the person skilled in the art such as, for example, evaporation, precipitation, extraction, passage over an ion exchange resin, electrodialysis, chromatographic methods, solidification of the hydrated inorganic salt by reducing the temperature or by adding a third substance, or reverse osmosis.

The additives which night be necessary for this step are introduced into the vessel 72 via the line 78.

At the outlet from the vessel 72, a liquid fraction 74 is obtained which contains the concentrated inorganic salt at least a portion f which is advantageously recycled to the digestion reactor 42 (step g). Optionally, water may be added to the stream 74 via the line 80 in order to adjust the water stoichiometry and obtain a hydrated inorganic salt with a composition which is identical to that introduced via the line 44. Preferably, the hydrated inorganic salt obtained has the same composition as that introduced via the line 44. Optionally, the liquid fraction 74 may contain all or a portion of the organic solvent.

The liquid fraction 76 which is depleted in inorganic salt may contain the anti-solvent, the organic solvent, the remainder of the products derived from the biomass and hydrated inorganic salt. Preferably, the liquid fraction 76 which is depleted in inorganic salt contains less than 50% of the hydrated inorganic salt initially present in the fraction 54. Still more preferably, the liquid fraction 76 which is depleted in inorganic salt contains less than 25% of the hydrated inorganic salt initially contained in the fraction 54.

When step h) is carried out with the addition of an anti-solvent, the anti-solvent is mainly recovered in the liquid fraction 76 which is depleted in inorganic salt and may be recycled (not shown) to the step h) after optional re-treatment, or to step i) in the case in which the FIG. 2 layout is operated.

In accordance with another variation, step i) for the treatment of the solid fraction obtained in step h) is carried out by one or more washes in order to obtain a treated solid fraction 62 and a liquid fraction 60, at least a portion of said liquid fraction being sent to a purification step 72 denoted step k), in order to concentrate the inorganic salt contained in the liquid fraction and to obtain a liquid fraction 74 containing the concentrated inorganic salt and another liquid fraction 76 which is depleted in inorganic salt, at least a portion of said liquid fraction 74 containing the concentrated inorganic salt then being recycled to the digestion step g). When step i) is carried out with the addition of an anti-solvent, any residues from this (these) treatment(s) are withdrawn via the line 60 then either purged at 84, or sent to the vessel 72.

In accordance with an embodiment which is not shown, the anti-solvent 58 added to step i) is separated during the purification step 72 and recycled to the step i).

1. A process for the production of solutions of sugars and alcohols from lignocellulosic biomass, comprising the following steps:
a) carrying out a pre-treatment of a feed comprising lignocellulosic biomass,
b) carrying out an enzymatic hydrolysis of the pre-treated and optionally washed feed obtained from step a) using cellulolytic and/or hemicellulolytic enzymes, producing an effluent comprising a hydrolysate containing sugars and a solid residue,
c) carrying out an alcoholic fermentation of the hydrolysate contained in the effluent obtained from step b) into alcohol with an alcoholigenic microorganism in order to produce a fermented effluent containing alcohol,
d) carrying out a step for extracting alcohol from the fermented effluent obtained from step c),
e) carrying out a step for separating the effluent obtained either at the end of step c) or at the end of step d) in order to produce a solid residue and a liquid fraction,
f) carrying out an optional step for washing and an optional step for drying the solid residue obtained in step e),
g) carrying out a step for digesting the solid residue obtained in step e) and/or f) in a medium comprising at least one hydrated inorganic salt with formula MXₙ.n′H₂O, in which M is a metal selected from groups 1 to 13 of the periodic classification, X is an anion and n is a whole number in the range 1 to 6 and n′ is in the range 0.5 to 12, in order to obtain a solid fraction and a liquid fraction,
h) carrying out a step for separating the solid fraction and the liquid fraction obtained in step g),
i) carrying out an optional step for treatment of said solid fraction obtained in step h),
j) carrying out either a step for enzymatic hydrolysis of said solid fraction obtained in step h) and/or i), or steps for enzymatic hydrolysis and alcoholic fermentation of said solid fraction obtained in step h) and/or i).

2. The process according to claim 1, in which the alcoholic fermentation of step c) and/or step j) produces ethanol.

3. The process according to claim 1, in which the enzymatic hydrolysis step b) and the alcoholic fermentation step c) are carried out separately or simultaneously.

4. The process according to claim 1, in which the pre-treatment is a thermal, chemical, mechanical and/or enzymatic treatment or a combination of these treatments.

5. The process according to claim 1, in which the pre-treatment of step a) is carried out under acid conditions or without chemical reagents, the feed which is thus pre-treated undergoing a separation step before step b) in order to recover a solid fraction and a liquid fraction, at least a portion of the solid fraction being sent to the enzymatic hydrolysis step b) and at least a portion of the liquid fraction being recycled to the enzymatic hydrolysis step j).

6. The process according to claim 1, in which the pre-treatment is carried out by steam explosion, with or without the addition of acid.

7. The process according to claim 1, in which at least a portion of the liquid fraction obtained in step e) is recycled to the enzymatic hydrolysis step j).

8. The process according to claim 1, in which at least a portion of the washing solution obtained in the step for washing the solid residue f) is recycled to the enzymatic hydrolysis step j).

9. The process according to claim 1 in which, in the digestion step g), the anion X of the hydrated inorganic salt with formula (I) is a halide anion selected from Cl⁻, F⁻, Br⁻ and I⁻, a perchlorate anion, a thiocyanate anion, a nitrate anion, an acetate anion, a para-methylbenzene sulphonate anion, a sulphate anion, an oxalate anion or a phosphate anion, and in which the metal M in formula (I) is selected from lithium, iron, zinc and aluminium.

10. The process according to claim 1 in which, in the digestion step g), the salt is selected from ZnCl₂.1.5H₂O, ZnCl₂.2.5H₂O and ZnCl₂.4H₂O.

11. The process according to claim 1, in which the digestion step g) is carried out in the presence of one or more organic solvents selected from alcohols such as methanol, ethanol, n-propanol, isopropanol, n-butanol, isobutanol or tert-butanol, diols and polyols such as ethanediol, propanediol or glycerol, aminoalcohols such as ethanolamine, diethanolamine or triethanolamine, ketones such as acetone or methylethylketone, carboxylic acids such as formic acid or acetic acid, dimethylformamide, dimethylacetamide, dimethylsulphoxide, acetonitrile and aromatic solvents such as benzene, toluene, xylenes and alkanes.

12. The process according to claim 1, in which the step h) for separation of the solid fraction is carried out by precipitation by adding at least one anti-solvent which is a solvent or a mixture of solvents selected from water, alcohols such as methanol, ethanol, n-propanol, isopropanol, n-butanol, isobutanol or tert-butanol, diols and polyols such as ethanediol, propanediol or glycerol, aminoalcohols such as ethanolamine, diethanolamine or triethanolamine, ketones such as acetone or methylethylketone, carboxylic acids such as formic acid or acetic acid, esters such as ethyl acetate or isopropyl acetate, dimethylformamide, dimethylacetamide, dimethylsulphoxide or acetonitrile.

13. The process according to claim 1, in which step i) for treatment of the solid fraction obtained in step h) is carried out by means of one or more washes, by neutralisation, by compression and/or by drying.

14. The process according to claim 1, in which at least a portion of the liquid fraction obtained in step h) is sent to a purification step, denoted step k), in order to concentrate the inorganic salt contained in the liquid fraction and obtain a liquid fraction containing the concentrated inorganic salt and another liquid fraction which is depleted in inorganic salt, at least a portion of said liquid fraction containing the concentrated inorganic salt then being sent to the digestion step g).

15. The process according to claim 1, in which step i) for treatment of the solid fraction obtained in step h) is carried out by means of one or more washes in order to obtain a treated solid fraction and a liquid fraction, at least a portion of said liquid fraction being sent to a purification step, denoted step k), in order to concentrate the inorganic salt contained in the liquid fraction and to obtain a liquid fraction containing the concentrated inorganic salt and another liquid fraction which is depleted in inorganic salt, at least a portion of said liquid fraction containing the concentrated inorganic salt then being recycled to the digestion step g).
